(19) United States
(12) Patent Application Publication
(10) Pub. No.: US 2022/0370831 A1
(43) Pub. Date: Nov. 24, 2022

(54) CONFORMITY INDEX EVALUATION TOOL AND METHOD FOR RADIOTHERAPY TREATMENT PLANNING

(71) Applicant: UNIVERSAL RADIOTHERAPY AR-GE SAGLIK HIZ. SAN. ve TIC. LTD. STI, Ankara (TR)

(72) Inventor: Levent GONULTAS, Ankara (TR)

(73) Assignee: UNIVERSAL RADIOTHERAPY AR-GE SAGLIK HIZ. SAN. ve TIC. LTD. STI, Ankara (TR)

(21) Appl. No.: 17/763,674

(22) PCT Filed: Sep. 4, 2020
(86) PCT No.: PCT/TR2020/050809

§ 371 Date: Mar. 25, 2022

(30) Foreign Application Priority Data
Sep. 27, 2019 (TR) 2019/14727

(51) Int. Cl. A61N 5/10

(52) U.S. Cl. CPC A61N 5/1071; A61N 5/103

(57) ABSTRACT

A dosimetric evaluation tool and method is used to determine how well the prescription isodose volume (PIV) conform to the size and shape both the tumor volume (TV) and the healthy tissue in radiotherapy treatment plans. The innovative, ideal, and universal dosimetric evaluation tools are Conformity Index (CI) and Unconformity Indexes (UCI_{underdose} and UCI_{overdose}). CI measures the conformity of the radiotherapy planning, and UCI_{underdose} and UCI_{overdose} measure the unconformity of the radiotherapy planning. In other words, UCI_{underdose} and UCI_{overdose} reflect the negative effect of dose distribution in planning, and CI reflects the positive effect of dose distribution.

### CROSS REFERENCE TO THE RELATED APPLICATIONS

This application is the national phase entry of International Application No. PCT/TR2020/050809, filed on Sep. 4, 2020, which is based upon and claims priority to Turkish Patent Application No. 2019/14727, filed on Sep. 27, 2019, the entire contents of which are incorporated herein by reference.

### TECHNICAL FIELD

The invention relates to dosimetric evaluation tools and method that uses to determine of how well the prescription isodose volume (PIV) conform to the size and shape both the tumor volume (TV) and the healthy tissue in radiotherapy treatment plans.

### BACKGROUND

Cancer patients are treated using ionized radiation in radiation oncology clinics. This treatment is called radiotherapy. Treatment is planned in the treatment planning system (TPS). Thus, irradiation conditions are determined.

First of radiotherapy aims at giving a high dose to the tumor volume but as low a dose as possible to the surrounding healthy tissue volume. Conformity Index is the dosimetric evaluation tool to use measuring of this aim.

Conformity index (CI) is a measure of how well the prescription isodose volume (PIV) conforms to the size and shape both the tumor volume (TV) and healthy tissue volume. That is, the ideal CI tool must reflect the negative effects on the conformity of the radiotherapy treatment plan. Negative effects are both the cold spots occurring in the TV and the irradiation of normal tissue and organs at risk (OAR) around the TV. There are two different using area of CI tool.
3. It is used as the evaluation tool to choose of the best dose distribution.
4. It is used as the optimization tool to create optimum plan

CI is the very important tool for evaluation and optimization of radiotherapy treatment plans but there is not ideal conformity index evaluation tool in literature. Various conformity indices have been proposed in the literature by different groups and scientists. The CI that was proposed in 1993 by the Radiation Therapy Oncology Groups (RTOG) and described in report 62 of the International Commission on Radiation and Measurements (ICRU) is as ratio of the PIV to the TV.

It is the most primitive form of CI expression. An RTOG CI greater than 1 indicates that the irradiated volume is greater than the TV and includes healthy tissues. If the CI is less than 1, the TV is only partially irradiated. However, this index presents a major drawback: It can never take into account the degree of spatial intersection of two volumes or their shapes. In extreme cases, it may be equal to 1 while these two volumes are situated away from each other and present entirely different shapes.

The Saint-Anne, Lariboisiere, Tenon (SALT) group proposed the lesion coverage volume factor (CVF) Lomax and Scheib also used this formula to measure CI. This SALT-Lomax CI is ratio of the TV_{PIV} (tumor volume covered by the PIV) to the TV.

According to Lomax and Scheib CVF represents the TV receiving at least the prescribed dose. The quality of irradiation of the TV can be correctly determined with CVF, but it does not provide sufficient information about the overall treatment plan.

Lomax and Scheib also proposed another CI formula that took irradiation of normal tissue and OARs into account is as ratio of the TVPIV to the PIV.

This index equal being to 1 may be very different from perfect conformation, because the prescription isodose volume can be totally included in the tumor volume, but part of the tumor volume may not be irradiated at the prescribed dose.

Van't Riet et al. proposed a CI called conformation number (CN) to measure CI. This CI is ratio of the TVPIV square to the TV times the PIV

According to Feuvret et al. the calculation of this CN simultaneously takes into account irradiation of the target volume and the irradiation of healthy tissues. The first fraction of this equation defines the quality of coverage of the tumor, the second defines the volume of healthy tissue receiving a dose greater than or equal to the prescribed reference dose.

Paddick proposed a CI formula that is ratio of the TVPIV square to the TV times the PIV

This is exactly equal to Van't Riet CI formula.

The deficiencies and shortcomings of the above formulas necessitate a new CI formula. Such a formula is discussed in detail in this specification.

### The Problems of Existing CI Tools

TABLE 1
Evaluating of CI equalities for the 5 different dose distributions that may occur as a result of planning

| Dose Distributions | Parameters (cm³) | $\begin{matrix}RTOG \\ CI = \frac{PIV}{TV}\end{matrix}$ | $\begin{matrix}SALT ‐ Lomax \\ CI = \frac{{TV}_{PIV}}{TV}\end{matrix}$ | $\begin{matrix}Lomax ‐ Scheib \\ CI = \frac{{TV}_{PIV}}{PIV}\end{matrix}$ | $\begin{matrix}Van ’ t Riet or \\ Paddick \\ CI = \frac{{TV}_{PIV}^{2}}{TV \times PIV}\end{matrix}$ |
|---|---|---|---|---|---|
| 1^{st} dose | TV = 8 | 1.25 | 1 | 0.8 | 0.8 |
| distribution | PIV = 10 | | | | |
| (FIG. 1) | TVₚᵢᵥ = 8 | | | | |
| 2^{nd} dose | TV = 8 | 0.75 | 0.75 | 1 | 0.75 |
| distribution | PIV = 6 | | | | |
| (FIG. 2) | TVₚᵢᵥ = 6 | | | | |
| 3^{rd} dose | TV = 8 | 1 | 0.5 | 0.5 | 0.25 |
| distribution | PIV = 8 | | | | |
| (FIG. 3) | TVₚᵢᵥ = 4 | | | | |
| 4^{th} dose | TV = 8 | 1 | 1 | 1 | 1 |
| distribution | PIV = 8 | | | | |
| (FIG. 4) | TVₚᵢᵥ = 8 | | | | |
| 5^{th} dose | TV = 8 | 1 | 0 | 0 | 0 |
| distribution | PIV = 8 | | | | |
| (FIG. 5) | TVₚᵢᵥ = 0 | | | | |

In the result of treatment planning of a cancer patient five different dose distributions may occur among the TV, PIV, and a volume of healthy tissue (normal tissue and OARs) that is irradiated unintentionally (Table 1). The conformity index is calculated as 1 (100%) for optimum condition of planning. But result of CI should approach zero when it increases the volume of healthy tissue that is irradiated unintentionally or the volume of cold spots in the TV that is undesired in radiotherapy treatment plans. These dose distributions are listed below.

First dose distribution: PIV may include the whole of the TV. In this condition, the whole of the TV is irradiated without any cold spots, but normal tissue and OARs are also irradiated. TVPIV becomes equal to TV. Normal tissue and OARs are irradiated without any cold spots in the TV in 1st dose distribution. However, CI should be less than 1 (100%) because the irradiation of normal tissue and OARs is undesired in radiotherapy treatment plans. Nevertheless, as seen in Table 1, the RTOG CI is equal to 1.25 (125%), which is confusing. Accordingly, the RTOG CI formula gives false results in plans where 1st dose distribution is valid. The SALT-Lomax CI formula is equal to 1, because the whole TV is covered by the PIV. This example illustrates the fundamental flaw of the SALT-Lomax CI: the irradiation of normal tissue and OARs around the TV is not taken into account. Other CI formulas give true results.

Second dose distribution: The whole of the PIV may remain inside the TV. In this condition, cold spots occur in the TV. TVPIV becomes equal to PIV. In 2nd dose distributions, CI should be less than 1 because the occurrence of cold spots in the TV is undesired in radiotherapy treatment plans. However, the Lomax and Scheib CI formula is equal to 1 (100%), which may be misinterpreted as perfect conformation. The CI formula proposed by Lomax and Scheib gives false results in plans where 2nd dose distribution is valid, because of cold spots in the TV (Table 1).

Third dose distribution: Although some parts of the TV remain inside the PIV, other parts of the TV may be outside the PIV. In this situation, cold spots occur in the TV and normal tissue, and OARs around the TV are irradiated.

In 3rd dose distribution, the RTOG CI, SALT-Lomax CI, and Lomax and Scheib CI formula give false results, because in this distribution, both normal tissue and OARs are irradiated as in 1st distribution, and cold spots occur in the TV as in 2nd dose distribution (Table 1). The SALT-Lomax CI was equal to 0.5, which shows that 50% of the TV was not irradiated. The Lomax and Scheib CI was equal to 0.5, which means that the volume of irradiated healthy tissues was 50% of the total irradiated volume. When the van't Riet or Paddick CI was equal to 0.25 (0.5×0.5), this was equal to the product of the SALT-Lomax CI and the Lomax and Scheib CI. The van't Riet CI and Paddick CI formulas give false results. These CI formulas are equal to the product of the CIs proposed by SALT-Lomax and Lomax and Scheib. However, These CIs never does not give the adequately information about the conformity of the treatment planning in 3rd dose distribution.

Fourth dose distribution: TV and PIV may completely match each other (target is missed completely), all CI formulas give 1 (perfect conformation) as expected (Table 1).

Fifth dose distribution: The TV and the PIV are situated distant from each other. This may occur in an algorithmic error for the treatment planning systems (TPS). In the 5th dose distribution, the TV and PIV are situated away from each other; therefore, the CI result is equal to 0 because all of the TV is outside the PIV and only normal tissue and OARs around the TV are irradiated. The RTOG CI is equal to 1 although it is far from perfect conformation. Other CI formulas give correct results (Table 1).

The results demonstrated that the RTOG CI only makes simple scoring about the conformity of a plan. The RTOG CI and SALT-Lomax CI formulae give true results only when the whole PIV remains inside the TV or normal tissue, and OARs around the TV are not irradiated. The Lomax and Scheib CI gives correct results only if the PIV covers the whole TV or cold spots do not occur in the TV. CN and Paddick CIs simultaneously take into account irradiation of the target volume and irradiation of healthy tissues.

### Technical Problems In Which the Invention Aims to Solve

The best choice of treatment plan could be based on the conformity index as well as DVH (Dose Volume Histogram). However, until now, an ideal and universal index did not exist that clearly indicated conformity by taking into account irradiation of healthy tissues as well as the TV.

The current problems in existing Conformity Indices (CIs) are summarized below:
1. There is no single algorithm to express the conformity of plans for each dose distributions. The algorithms available in the literature give inaccurate results when cold spots occur in the TV while healthy tissues are irradiated. So, there is not an ideal method for calculating a conformity index (CI). (Ideal: Completely compatible with the accepted definitions of the conformity) Existing CI formulas were neither sufficient nor applicable under all possible dose distributions. Some CIs only take into account the irradiation of healthy tissues, whereas others solely consider irradiation of the tumor.
2. Existing CI algorithms are not universally applicable. When CI is equal to 0.8, this does not mean that the conformity of plan is the 80%. So, results of CI should be in the range of 0 to 1, where 0 corresponds to a completely non-conforming irradiation, and 1 corresponds to a completely conforming irradiation.
3. Reasons of unconformity are not known. For example, the conformity of plan is 80%. What does the remaining 20% mean? This is currently not well understood. For example, do cold spots in TV or the irradiation of healthy tissues contribute? So, what do users do for the better plan.

Due to the problems of existing CI algorithms, planning results can only be evaluated with DVH (Dose Volume Histogram) in the present. This blocks to choose the better treatment plan for patient. This condition increases the probability of the following cases.
Tumor Control may decrease therefore recurrent in TV increases.
Irradiated healthy tissue may increase therefore complication of healthy tissue increases.
Low dose region in healthy tissue may increase therefore seconder cancer risk increases.

### SUMMARY

This invention has the following goals:
1. This invention is a universal applicable. This allows to compare of the plans with each other the in the world.
2. It is completely compatible with the accepted definitions of the conformity. So, it is an ideal evaluation tool.
3. Reason of unconformity is detected with the UCI_{underdose} and UCI_{overdose} algorithms.
4. Duration of treatment planning will be the less than the present.
5. So, our product will solve the problems of existing CI.
6. In addition to, a new evaluation method has been created using these evaluation tools (CI, UCT_{underdose} and UCI_{overdose}).

This invention includes 3 different interconnected dosimetric evaluation tools that eliminate all existing problems in existing CI evaluation tools. Thanks to this feature of the inventions, radiotherapy treatment for cancer patients will provide significant advantages in terms of better treatment of the disease with use CI evaluation method.

These advantages include:
d. Tumor Control increases, therefore recurrent in TV decreases.
e. Irradiated healthy tissue decreases therefore complication of healthy tissue decreases.
f. Low dose region in healthy tissue decreases therefore seconder cancer risk decreases.

### BRIEF DESCRIPTION OF THE DRAWINGS

FIG. 1 shows the first dose distribution.

FIG. 2 shows the second dose distribution.

FIG. 3 shows the third dose distribution.

FIG. 4 shows the fourth dose distribution.

FIG. 5 shows the fifth dose distribution.

FIG. 6 shows the simulation of the AUB (the union of the A and B) and the A\B (A difference B) used in mathematics for PIV and TV.

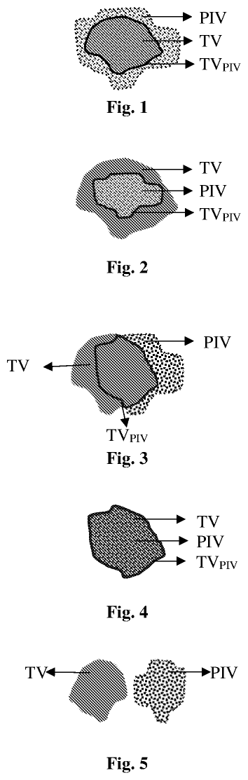

### DETAILED DESCRIPTION OF THE EMBODIMENTS

To eliminate these first and second problems mentioned in the technical problems section where the invention aims to solve, the CI must measure the proportion of the positive effect to the total of the negative and positive effects in the dose distribution as a result the treatment plan.
The negative effect is that part of the TV is not irradiated, and healthy tissues are irradiated.
The positive effect is that part of the TV is irradiated.

The TV covered by the PIV (TV_{PIV}) reflected positive effect of a treatment plan has already been defined in the literature. However, a new volume formed by the union of the TV and PIV (overall treatment plans) is needed. This volume must reflect the total effect of the treatment plan for each dose distribution. That is, the CI must measure the proportion of TV_{PIV} to this new volume to give the conformity of a plan with 100% agreement.

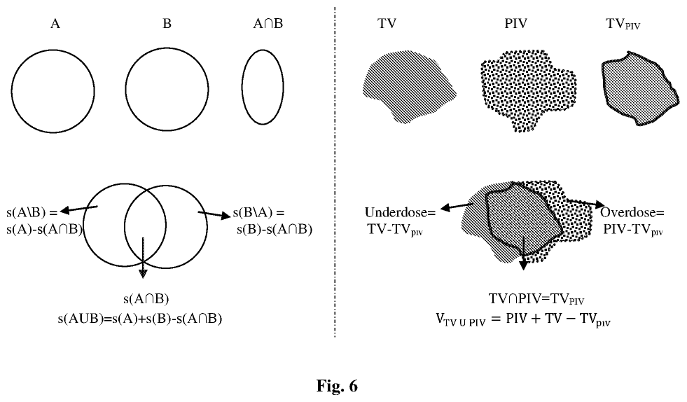

This new volume called V_{TV∪PIV} can be written with the union formula used in mathematics (FIG. 6).

V_{TV∪PIV}=PIV+TV−TV_{PIV} (1)

Where V_{TV∪PIV}=volume formed by union of TV and PIV.

Thus, conformity of a plan can be expressed as:

$$CI = \frac{T {V}_{PIV}}{{V}_{TV ⋃ PIV}} = \frac{T {V}_{PIV}}{T V + P I V - T {V}_{PIV}} \qquad ( 2 )$$

Also, to eliminate the last problem mentioned in the technical problems section where the invention aims to solve, new expressions supporting the ideal CI expression should be derived. In this invention, UCI_{underdose} (Unconformity Index created by cold spots remaining in tumor volume) and UCI_{overdose} (Unconformity Index formed by dose of healthy tissues) will be dosimetric evaluation tools.

To interpret the result of the conformity index, we also need to measure the effect on the CI of underdosing the tumor and the overdosing healthy tissues. These UCI_{underdose} and the UCI_{overdose} equalities can be described with the difference formula used in mathematics: (FIG. 6)

$${UCI}_{underdose} = \frac{T V - T {V}_{PIV}}{T V + P I V - T {V}_{PIV}} \qquad ( 3 )$$

$${UCI}_{overdose} = \frac{P I V - T {V}_{PIV}}{T V + P I V - {TV}_{PIV}} \qquad ( 4 )$$

CI measures the conformity of planning, and UCI_{overdose} and UCI_{underdose} measure the unconformity of planning. In other words, UCI_{overdose} and UCI_{underdose} reflect the negative effect of dose distribution in planning, and CI reflects the positive effect of dose distribution. This is correct, because the sum of CI, UCI_{overdose} and UCI_{underdose} are equal to 1, as shown below:

$$CI + {UCI}_{underdose} + {UCI}_{overdose} = 1 \frac{{TV}_{PIV}}{T V + P I V - {TV}_{PIV}} + \frac{TV - {TV}_{PIV}}{T V + P I V - {TV}_{PIV}} + \frac{P I V - {TV}_{PIV}}{T V + P I V - {TV}_{PIV}} = \frac{T V + P I V - {TV}_{PIV}}{T V + P I V - {TV}_{PIV}} = 1$$

After the patient's treatment plan was established, CI and UCI_{overdose} and UCI_{underdose} evaluation tools are calculated. If the CI result complies with clinical protocols, the patient is taken into treatment through the relevant treatment planning. If the results do not comply with clinical protocols results, UCI_{overdose} and UCI_{underdose} are evaluated. According to these results, the cause of unconformity is determined. Finally, new treatment plans are created that eliminate this cause. CI should re-evaluate for this new treatment plan and if the result complies with clinical protocols, the patient is treated with the relevant plan.

What is claimed is:

1. A Conformity Index (CI) evaluation method, wherein the CI evaluation method utilizes a CI tool, an UCI_{underdose} tool, and a UCI_{overdose} tool in an iterative manner in order to evaluate a radiotherapy treatment plan for radiotherapy treatment planning, and the CI evaluation method comprises the following steps:
1) measuring a conformity of a dose distribution to a radiotherapy target,
2) determining if the dose distribution is successful with the CI tool;
3) determining a cause of an unconformity of the dose distribution;
4) measuring a magnitude of the unconformity of the dose distribution with the UCI_{underdose} tool and UCI_{overdose} tool.

2. The CI evaluation method according to claim 1, wherein to determine how well a prescription isodose volume (PIV) conforms to a size and a shape of both a tumor volume (TV) and a healthy tissue volume, step 1 comprises:
measuring a proportion of a positive effect to V_{TV∪PIV} in the dose distribution as a result the radiotherapy treatment plan; and
calculating a proportion of TV_{PIV} to V_{TV∪PIV};
wherein
the negative effect is when a part of the tumor volume is not irradiated, and a part of a healthy tissue is irradiated, and the positive effect is when the tumor volume is irradiated;
V_{TV∪PIV} is a total of the negative effect and the positive effect in the dose distribution as the result of the radiotherapy treatment plan;
TV_{PIV} is a tumor volume covered by the prescription isodose volume.

3. The CI evaluation method according to claim 2, wherein V_{TV∪PIV} reflects a total effect of the radiotherapy treatment plan for each of the dose distribution, and V_{TV∪PIV} is determined by the following formula:
V_{TV∪PIV}=PIV+TV−TV_{PIV}.

4. The CI evaluation method according to claim 1, wherein the UCI_{underdose} tool is a measure of the unconformity of the dose distribution in the radiotherapy treatment plan, and the CI evaluation method comprises the step of calculating UCT_{underdose} with the following formula to measure a negative effect of underdosing a tumor on the conformity of the dose distribution:
$$\text{?}$$
$$\text{?} \text{indicates text missing or illegible when filed}$$
wherein
the negative effect is when a part of the tumor volume is not irradiated, and a part of a healthy tissue is irradiated;
PIV is a prescription isodose volume;
TV is a tumor volume;
TV_{PIV} is a tumor volume covered by the prescription isodose volume.

5. The CI evaluation method according to claim 1, wherein the UCI_{overdose} tool is a measure of the unconformity of the dose distribution in the radiotherapy treatment plan, and the CI evaluation method comprises the step of calculating UCI_{overdose} with the following formula to measure a negative effect of overdosing a healthy tissue on the conformity of the dose distribution:
$$\text{?}$$
$$\text{?} \text{indicates text missing or illegible when filed}$$
wherein
the negative effect is when a part of the tumor volume is not irradiated, and a part of the healthy tissue is irradiated;
PIV is a prescription isodose volume;
TV is a tumor volume;
TV_{PIV} is a tumor volume covered by the prescription isodose volume.
